(19) United States
(12) Patent Application Publication
(10) Pub. No.: US 2026/0127177 A1
(43) Pub. Date: May 7, 2026

(54) CLINICAL PROCESSING AUTOMATION USING RELATIONAL MODELING OF ATOMIC DOCUMENT ELEMENTS

(71) Applicant: Cohere Health, Inc., Boston, MA (US)

(72) Inventors: Jackson Mostoller, Surprise, AZ (US); Parth Anand Jawale, Seattle, WA (US); Isaac Lo, San Francisco, CA (US); Ben Barone, West Fulton, NY (US)

(73) Assignee: Cohere Health, Inc., Boston, MA (US)

(21) Appl. No.: 19/376,367

(22) Filed: Oct. 31, 2025

Related U.S. Application Data
(60) Provisional application No. 63/715,425, filed on Nov. 1, 2024.

(51) Int. Cl. G06F 16/2455; G06F 16/28; G16H 10/20

(52) U.S. Cl. CPC G06F 16/24553; G06F 16/284; G16H 10/20

(57) ABSTRACT

A system can establish a database that includes a plurality of atomic units from documents relating to one or more clinical actions. The system can receive a request for authorization of a clinical action and determine, using a rules engine, one or more rules for a type of the clinical action. The system can generate a query to the relational database to retrieve, from the relational database, a group of atomic units dynamically identified as corresponding to the type of chunk and based on the one or more filters. The system can determine, by the rules engine using the one or more rules, that the group of atomic units resulting from the query satisfy the one or more rules. The system can authorize the clinical action responsive to the determination.

### CROSS-REFERENCE TO RELATED APPLICATIONS

The present application claims the benefit of and priority to U.S. Provisional Application No. 63/715,425, filed Nov. 1, 2024, the disclosure of which is incorporated herein by reference in its entirety.

### BACKGROUND

Information retrieval systems are used to manage, store, and retrieve large volumes of digital data from diverse sources. Unstructured data such as text, images, audio, and other multimedia formats often require specialized tools for processing and searching. However, existing systems face difficulties in handling heterogeneous data types, maintaining metadata consistency, and enabling efficient retrieval across different modalities. This can lead to retrieval that lacks in performance in speed, compute requirements, and/or data storage requirements. In the context of automation of clinical processes, retrieving relevant data for the automation can be constrained by such limitations, such as to reduce the speed in which systems can perform automation, or to require significant amounts of data storage and/or persistence to facilitate functions such as report generation or maintaining data for audit trails.

### SUMMARY

Systems and methods in accordance with the present disclosure can represent documents and their components as relational data, including by extracting atomic units of data in any of a variety of modalities, and grouping, e.g., chunking, the atomic units into chunks to respond to queries for data retrieval. For example, the system can provide dynamic view-based chunking in which the chunks are provided as views over the atomic units, rather than relying on chunks that are fixed at indexing of the documents. This can allow for variable granularity of retrieval without re-indexing. Metadata, including spatial and semantic annotations, can be associated with atomic units directly, and can be aggregated at the chunk level through relational joins or grouping operations. In response to a query, retrieval operations can be expressed as composable relational expressions that select, filter, or aggregate atomic and chunk-level attributes from a unified multimodal corpus. This can allow for flexible and consistent information access across different data types. The system can allow for multi-stage retrieval operations, which can allow for more efficient retrieval of relevant data. For example, systems and methods as described herein can achieve faster retrieval, including with fewer requirements for intermediate data to be stored or maintained. Systems and methods in accordance with the present disclosure can be applied to retrieval tasks in any of a variety of applications, including but not limited to document generation or processing, classification, clinical workflows, administrative workflows, healthcare operations including prior authorization, scheduling, patient support, clinician support, claims processing, chart or lab processing, report generation, conversational agent management, or various combinations thereof.

The techniques described herein can represent clinical documents and their constituent elements as relational data structures, where each atomic unit of data extracted from a document (such as a token or pixel) can be stored as a record having content attributes and corresponding metadata. In some implementations, atomic-level data can be grouped into chunks using dynamically defined views that can be modified without re-indexing. Chunk definitions can be expressed as relational expressions across atomic tables, such that retrieval operations can be evaluated directly as joins, filters, or aggregations within a unified corpus. Metadata describing temporal, spatial, or semantic context can be associated at the atomic level and propagated to chunk-level groupings by aggregation operations. In some implementations, queries issued in response to a clinical request can reference atomic and chunk-level attributes through composable relational expressions that can be used to retrieve or evaluate relevant information for automated decision making. Such relational modeling of atomic document elements can support multimodal and multi-stage retrieval workflows for prior authorization processing, claims processing, audit record generation, and other clinical automation tasks.

At least one aspect relates to a system. The system can receive a plurality of documents comprising unstructured data. The system can determine a type of modality for each document of the plurality of documents. The system can route each document to a corresponding parser based on the type of modality for the document. The system can select an atomic unit type for parsing each document based on the type of modality. The system can parse at least the unstructured data of each document according to the atomic unit type to extract a plurality of atomic units from the document and a plurality of attributes of each atomic unit. The system can update a table in a relational database to include a record for each atomic unit, the record including a unique identifier of the atomic unit, a document identifier linking the atomic unit to the document from which the atomic unit is extracted, and the plurality of attributes of the atomic unit. The system can output, in response to a request for a chunk of one or more atomic units, at least one record corresponding to the chunk, where the chunk is dynamically defined responsive to the request.

In some implementations, the system can dynamically define the chunk as a selection of one or more atomic units based on one or more criteria indicated by the request. In some implementations, the system can represent the chunk as a first table comprising one or more chunk-level attributes of the chunk and a second table comprising an identifier of the chunk and the unique identifier of each atomic unit of the chunk. In some implementations, the system can output the chunk, based on the request, to include atomic units of a plurality of modalities. In some implementations, the request can be a first request indicating one or more first criteria for selection of atomic units, and the system can output responsive to a second request indicating one or more second criteria, a subset of the atomic units of the chunk. In some implementations, the system can provide, for generation of the request, a function to select atomic units according to a content attribute or a metadata attribute of the atomic units. In some implementations, the system can output the record to include both text data and image data. In some implementations, the system can generate the plurality of attributes of each atomic unit to include a location of the atomic unit in the document from which the atomic unit is extracted. In some implementations, the plurality of documents can include a plurality of modalities including at least a text modality and an image modality. In some implementations, the system can determine that the plurality of attributes of each atomic unit include at least one of a text value or a pixel color of the atomic unit and at least one of a position or a time stamp of the atomic unit. In some implementations, the atomic unit type can include a text token type, an image pixel type, or an audio sample type, and the system can use the corresponding parser to perform tokenization, pixel identification, or audio sampling of the document. In some implementations, the system can determine, based on the request, at least one of a relevance score, an embedding, a text representation, or a bounding box for the chunk.

At least one other aspect relates to a method. The method can be performed, for example, by one or more processors coupled to non-transitory memory. The method can include receiving a plurality of documents comprising unstructured data. The method can include determining a type of modality for each document of the plurality of documents. The method can include routing each document to a corresponding parser based on the type of modality for the document. The method can include selecting an atomic unit type for parsing each document based on the type of modality. The method can include parsing at least the unstructured data of each document according to the atomic unit type to extract a plurality of atomic units from the document and a plurality of attributes of each atomic unit. The method can include updating a table in a relational database to include a record for each atomic unit, the record including a unique identifier of the atomic unit, a document identifier linking the atomic unit to the document from which the atomic unit is extracted, and the plurality of attributes of the atomic unit. The method can include outputting, in response to a request for a chunk of one or more atomic units, at least one record corresponding to the chunk, the chunk being dynamically defined responsive to the request.

In some implementations, the method can include defining the chunk as a selection of one or more atomic units based on one or more criteria indicated by the request. In some implementations, the method can include structuring the chunk as a first table comprising one or more chunk-level attributes of the chunk and a second table comprising an identifier of the chunk and the unique identifier of each atomic unit of the chunk. In some implementations, the request can be a first request indicating one or more first criteria for selection of atomic units, and the method can include outputting responsive to a second request indicating one or more second criteria, a subset of the one or more atomic units of the chunk. In some implementations, the method can include providing for generation of the request a function to select atomic units according to a content attribute or a metadata attribute of the atomic units. In some implementations, the method can include generating the plurality of attributes of each atomic unit to include a location of the atomic unit in the document from which the atomic unit is extracted. In some implementations, the method can include determining that the plurality of attributes of each atomic unit include at least one of a text value or a pixel color of the atomic unit and at least one of a position or a time stamp of the atomic unit. In some implementations, the atomic unit type can include a text token type, an image pixel type, or an audio sample type, and the method can include using the corresponding parser to perform tokenization, pixel identification, or audio sampling of the document.

At least one aspect relates to a non-transitory computer-readable medium. The non-transitory computer-readable medium includes machine-readable instructions that when executed by one or more processors, cause the one or more processors to execute operations including parsing one or more documents, according to one or more modalities of the one or more documents, to extract a plurality of atomic units from the one or more documents and a plurality of attributes of each atomic unit of the plurality of atomic units; updating a table in a relational database to include a record for each atomic unit of the plurality of atomic units, the record comprising a unique identifier of the atomic unit, a document identifier linking the atomic unit to the document from which the atomic unit is extracted, and the plurality of attributes of the atomic unit; and outputting, based at least on a request for a chunk of one or more atomic units, at least a portion of at least one record corresponding to the chunk.

At least one aspect relates to a system. The system can establish a relational database that includes a plurality of atomic units extracted from a plurality of documents relating to one or more clinical actions, based at least on a type of modality of each document. The system can receive a request for authorization of a clinical action. The system can determine, using a rules engine, one or more rules for a type of the clinical action. The system can generate a query to the relational database, the query comprising a type of chunk relevant to the type of the clinical action and one or more filters for the query. The system can input the query to the relational database to retrieve, from the relational database, a group of atomic units dynamically identified as corresponding to the type of chunk and based on the one or more filters. The system can determine, by the rules engine using the one or more rules, that the group of atomic units resulting from the query satisfy the one or more rules. The system can authorize the clinical action responsive to the determination.

In some implementations, the system can select at least one of the rules engine or the one or more rules based on at least one of the type of the clinical action or the group of atomic units. In some implementations, the system can formulate the one or more filters for the query to include at least one filter related to the type of the clinical action. In some implementations, the system can retrieve the group of atomic units to include atomized content from one or more documents of the plurality of documents from which the group of atomic units are extracted and metadata of the group of atomic units. In some implementations, the system can generate, using the rules engine, a candidate determination that the group of atomic units resulting from the query satisfy the one or more rules. In some implementations, the system can present, using a user interface, an indication of the candidate determination. In some implementations, the system can receive, via the user interface, a confirmation of the candidate determination. In some implementations, the system can authorize the clinical action based on the confirmation.

In some implementations, the system can extract, from a given document of the plurality of documents, each of a first atomic unit comprising a token representing text and a second atomic unit comprising a pixel of an image. In some implementations, the system can assign a first position attribute to the first atomic unit indicating a position of the text in the given document. In some implementations, the system can assign a second position attribute to the second atomic unit indicating a position of the pixel in the document. In some implementations, the system can define the one or more filters to select one or more medical records regarding a patient for which to authorize the clinical action. In some implementations, the clinical action can comprise at least one of a test to perform for a patient, a treatment to provide to the patient, or an appointment to schedule between the patient and a provider.

In some implementations, the plurality of documents can comprise at least one of a medical record, diagnostic imaging data, a test result, or claims data. In some implementations, the plurality of documents can comprise at least one of a facsimile document or a portable document format document. In some implementations, the plurality of documents can comprise a guideline document regarding the clinical action. In some implementations, the system can select the one or more rules according to an atomic unit corresponding to the guideline document. In some implementations, the system can generate the query, according to the one or more rules, to select at least a portion of the guideline document. In some implementations, the one or more rules can identify the one or more filters. In some implementations, the system can generate audit data regarding the determination, the audit data comprising content of at least one atomic unit of the group of atomic units and a location of the content in a corresponding document of the plurality of documents from which the at least one atomic unit is extracted.

At least one other aspect relates to a method. The method can be performed, for example, by one or more processors coupled to non-transitory memory. The method can include receiving a request for authorization of a clinical action. The method can include determining, using a rules engine, one or more rules for a type of the clinical action. The method can include generating a query to a relational database that includes a plurality of atomic units extracted from a plurality of documents, the query comprising a type of chunk relevant to the type of the clinical action and one or more filters for the query. The method can include inputting the query to the relational database to retrieve a group of the plurality of atomic units, the group dynamically identified as corresponding to the type of chunk and based on the one or more filters. The method can include determining, by the rules engine using the one or more rules, that the group of atomic units resulting from the query satisfy the one or more rules. The method can include authorizing the clinical action responsive to the determination.

In some implementations, the method can include receiving the request from a clinical system remote from the one or more processors, and transmitting an indication of the authorization to the clinical system. In some implementations, the method can include generating, using the rules engine, a candidate determination that the group of atomic units resulting from the query satisfy the one or more rules. In some implementations, the method can include receiving, via a user interface, a confirmation of the candidate determination. In some implementations, the method can include outputting the authorization of the clinical action based on the confirmation. In some implementations, the method can include generating the one or more filters, based on the one or more rules, to query for notes regarding a previous clinical interaction with a patient associated with the request and for a protocol for the clinical action. In some implementations, the method can include generating the one or more filters to select one or more medical records regarding a patient for which to authorize the clinical action. In some implementations, the plurality of documents can comprise at least one of a medical record, diagnostic imaging data, a test result, or claims data.

At least one other aspect relates to a non-transitory computer-readable medium. The non-transitory computer-readable medium can include machine-readable instructions that, when executed by one or more processors, cause the one or more processors to update a relational database to include a plurality of atomic units extracted from a plurality of documents relating to a clinical action, based at least on a type of modality of each document. The machine-readable instructions can cause the one or more processors to receive a request for authorization of the clinical action. The machine-readable instructions can cause the one or more processors to determine, using a rules engine, one or more rules for a type of the clinical action. The machine-readable instructions can cause the one or more processors to generate a query to the relational database, the query comprising a type of chunk relevant to the type of the clinical action and one or more filters for the query. The machine-readable instructions can cause the one or more processors to input the query to the relational database to retrieve, from the relational database, a group of atomic units dynamically identified as corresponding to the type of clinical action and based on the one or more filters. The machine-readable instructions can cause the one or more processors to determine, by the rules engine using the one or more rules, that the group of atomic units resulting from the query satisfy the one or more rules. The machine-readable instructions can cause the one or more processors to transmit an authorization of the clinical action responsive to the determination.

These and other aspects and implementations are discussed in detail below. The foregoing information and the following detailed description include illustrative examples of various aspects and implementations and provide an overview or framework for understanding the nature and character of the claimed aspects and implementations. The drawings provide illustration and a further understanding of the various aspects and implementations and are incorporated in and constitute a part of this specification. Aspects can be combined, and it will be readily appreciated that features described in the context of one aspect of the invention can be combined with other aspects. Aspects can be implemented in any convenient form, for example, by appropriate computer programs, which may be carried on appropriate carrier media (computer readable media), which may be tangible carrier media (e.g., disks) or intangible carrier media (e.g., communications signals). Aspects may also be implemented using any suitable apparatus, which may take the form of programmable computers running computer programs arranged to implement the aspect. As used in the specification and in the claims, the singular form of ‘a,’ ‘an,’ and ‘the’ include plural referents unless the context clearly dictates otherwise.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 is a block diagram illustrating an example of an atomic relational retrieval system, in accordance with one or more implementations;

FIG. 2 is a flow diagram of an example of an atomized relational retrieval process, in accordance with one or more implementations;

FIG. 3 is a flow diagram of an example of an atomized relational retrieval process, in accordance with one or more implementations; and

FIG. 4 is a flow chart illustrating a method of atomized relational retrieval, in accordance with one or more implementations.

FIG. 5 is a block diagram of an automated authorization system for processing clinical requests, in accordance with one or more implementations;

FIG. 6 is a block diagram illustrating a clinical decision making workflow, in accordance with one or more implementations; and

FIG. 7 is a flow chart illustrating a method for authorizing a clinical action using relational retrieval of atomic units, in accordance with one or more implementations.

### DETAILED DESCRIPTION

Below are detailed descriptions of various concepts related to, and approaches, methods, apparatuses, and systems for implementing the various techniques described herein. The various concepts introduced above and discussed in greater detail below may be implemented in any of numerous ways, as the described concepts are not limited to any particular manner of implementation. Examples of specific implementations and applications are provided primarily for illustrative purposes.

The present disclosure relates to techniques for representing unstructured or multimodal data in a relational format to enable flexible and fine-grained information retrieval. Data used in information retrieval systems can originate from diverse document types such as text, images, and audio recordings. Each type of data can include different structures, metadata, and content attributes, which can require the use of specific parsers or processing tools to extract information suitable for downstream retrieval tasks. Conventional information retrieval systems can operate on a document-level or file-level representation. Relational database management systems can, by contrast, provide structured access to tabular data with clear definitions for relationships, indexes, and attributes.

Existing information retrieval architectures can encounter technical challenges when managing heterogeneous data or performing search operations that rely on context-specific representations. For example, traditional indexing systems can create static indexes that depend on predetermined segmentation strategies or token-level splits. This can include, for example, relying on chunks defined at index time. When retrieval tasks require different chunk sizes or new relevance metrics, many systems must be rebuilt from scratch to accommodate the new configuration. Metadata such as positional coordinates, timestamps, or semantic annotations can become fragmented across different data stores, complicating relational queries. Furthermore, retrieval operations that span multiple modalities, such as combining text relevance with visual similarity, can require disparate processing pipelines, which can increase computational overhead, and can limit consistency of results across modalities.

The techniques described herein can address any of various such challenges by implementing a relational representation of unstructured information, such as at a granular level. For example, the system can extract atomic units from documents, such as tokens for text, pixels for images, or samples for audio. Each atomic unit can retain attributes such as identifiers, positional data, semantic embeddings, and/or other metadata fields. The system can store the atomic units as relational records, and can dynamically group atomic units into higher-level constructs such as chunks (e.g., groups of atomic units and/or data thereof). The system can define or represent chunks as relational views or expressions that reference subsets of atomic units according to selection criteria or specific application needs. Retrieval can therefore occur at variable levels of granularity without requiring re-indexing of the original corpus.

In some implementations, the system can maintain and/or update a relational storage structure that includes one or more tables representing atomic units (and/or attributes of atomic units) and one or more tables representing chunks (and/or attributes of chunks). The system can parse documents into atomic units based on modality-specific parsing logic, such as to use one or more parsers that correspond to the type(s) of modalities of the documents. Each atomic unit can be stored as a record in a relational table with unique identifiers and associated attributes. A retrieval component can transform user queries into relational expressions that filter, join, and/or aggregate the stored atomic records to reconstruct relevant chunks. Additional components can enrich chunks with derived attributes, such as relevance scores or embedding-based similarity metrics. In some implementations, because the system can define chunks as views rather than static entities, the same dataset can support multiple retrieval strategies without altering the underlying data (including, for example and without limitation, performing retrieval based on both page chunks and sentence chunks).

By applying relational modeling principles to unstructured data, the techniques described herein can provide significant technical improvements over conventional information retrieval pipelines. These improvements can include a unified representation that preserves all metadata as first-class query-accessible fields, dynamic and non-destructive chunking that eliminates the need for re-indexing, and/or the ability to integrate multimodal relevance signals within a single query framework. As a result, retrieval workloads can operate more efficiently, perform queries across multiple modalities with consistent semantics, and/or maintain precise traceability between retrieved chunks and the original atomic data. The system can apply atomic-level storage and dynamic relational retrieval to provide a more expressive and/or flexible foundation for multimodal information access.

Systems and methods in accordance with the present disclosure can represent unstructured clinical information in a relational data format such that heterogeneous digital content can be efficiently analyzed and retrieved for healthcare applications. Clinical data can arise from multiple modalities, such as electronic health records, medical imaging, claims documents, laboratory reports, and faxed attachments. Each source of data can include content and metadata attributes that describe clinical context, spatial information, or temporal sequence. Relational database systems and traditional information retrieval frameworks have separately provided structured access methods for data management, yet these tools have historically been applied to distinct classes of data, with relational databases applied to structured records and information retrieval models applied to unstructured documents.

Conventional clinical automation and decision support platforms can face challenges when integrating diverse data formats or changing retrieval requirements. Existing approaches frequently index entire documents as single textual or image entities, which can limit fine-grained access to relevant content. When a change in clinical logic or rule evaluation requires new retrieval parameters, entire datasets may require re-indexing, which can increase processing latency and storage overhead. Metadata such as imaging coordinates, timestamps, or diagnostic annotations can be fragmented across data stores, which can complicate the linkage between clinical content and contextual features required for automated review or prior authorization workflows.

The techniques described herein provide a relational approach for representing unstructured clinical information such that each smallest data element, referred to as an atomic unit, can be stored as a record with defined attributes, including metadata and positional data. Atomic units can be grouped dynamically into relational views referred to as chunks, which represent retrieval units such as paragraphs, image regions, or diagnostic sections. Queries for rule-based decision engines can operate directly on these relational structures, allowing clinical authorization, audit record generation, and multimodal reasoning to occur without re-indexing. By structuring atomic content and metadata as first-class relational entities, clinical information retrieval can occur with improved granularity, reduced latency, and consistent interoperability across medical text, imaging, and structured datasets. This can be used, for example and without limitation, in claims processing, prior authorization, and clinical authorization tasks.

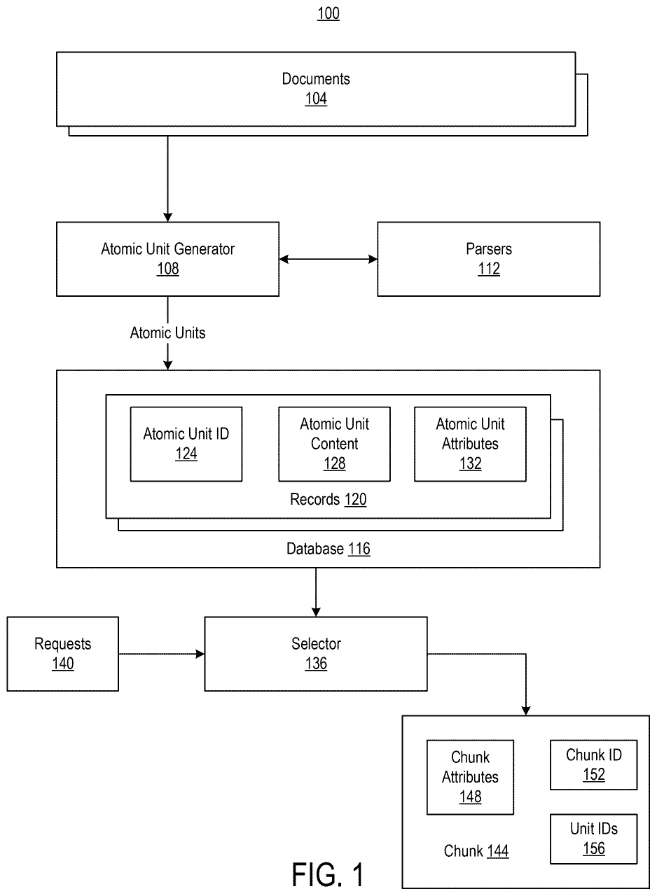

Referring now to FIG. 1, illustrated is a block diagram of an example system 100, such as an information retrieval system 100, in accordance with one or more implementations. The system 100 can perform retrieval of data from unstructured or multimodal sources using relational representations. For example, the system 100 can execute a retrieval pipeline for documents in any of a plurality of modalities or multiple modalities, such as any one or more of text, speech, audio, image, and/or video modalities. The system 100 can include or be operated using any of various computing hardware and/or software components, including but not limited to central processing unit (CPU) and/or graphics processing unit (GPU) systems. The system 100 can include one or more hardware and/or software components to execute operations described herein, such as one or more processors, hardware, software, databases, algorithms, functions, modules, neural networks, machine learning models, heuristics, policies, rules, or various combinations thereof. The system 100 can be structured as or to operate on any of various computing architectures, including, for example and without limitation, an on-premises system, a cloud-based system, a client-server architecture, a data center-based architecture, or various combinations thereof. The system 100 can handle retrieval for any of a variety of tasks, including but not limited to retrieval-based processes for language models, vision-language models or other vision or multimodal models, document generation or processing, classification, clinical workflows, administrative workflows, prior authorization, scheduling, patient support, clinician support, claims processing, chart or lab processing, report generation, conversational agent management, or various combinations thereof.

The system 100 can include or be coupled with at least one source of documents 104. The documents 104 can represent input content of various modalities, including text, speech, images, video, and audio. The documents 104 can be electronic data files. In some implementations, the documents 104 may include heterogeneous files of differing structures or encodings that require distinct parsing logic. For example, a corpus of digital files such as PDFs, scanned pages, or recorded signals can serve as the documents 104, and each file type may provide metadata indicating its structure or format. The documents 104 can include unstructured information, such as textual, visual, or temporal elements without predefined schema. In some implementations, each document 104 can include information of multiple modalities, such as text embedded within images or audio tracks accompanied by timestamped textual annotations, which the system 100 can process to extract distinct atomic units corresponding to each modality. In some implementations, the system 100 can receive and/or structure the documents 104 as a corpus of documents 104 (e.g., as described further herein, to structure the documents 104 as a collection of atomic units).

The system 100 can receive the documents 104 through a data ingestion interface. The system 100 can store references to each file in association with identifying attributes, such as file name, modality indicator, or source identifier. The system 100 can include or implement any of various database management components, including but not limited to SQL or functionality analogous to SQL, to facilitate data ingestion, processing, storing, and/or retrieval.

The system 100 can include an atomic unit generator 108. The atomic unit generator 108 can extract atomic units of data (e.g., atoms of data) from any one or more documents 104. The atomic units can be portions of the data of the documents 104, such as portions of the unstructured data of the documents 104. The system 100 can generate the atomic units to include or represent content from the documents 104. The system 100 can generate the atomic units to collectively represent all of the data of the documents 104, or subset of the data of the documents 104.

Each atomic unit can have an atomic unit type. The atomic unit type can correspond to a type of the data of the atomic unit. For example, the atomic unit type can include a text type, such as text tokens, or words, sentences, or paragraphs; an image and/or video type, such as pixels (or blocks or other groups of pixels); or an audio and/or speech type, such as samples of audio, such as segments of audio. For example, the atomic unit generator 108 can generate, from a given document 104, a plurality of atomic units having atomic unit types that correspond to the types of modalities of the given document 104.

In some implementations, the system 100 can include or be coupled with one or more parsers 112. The parsers 112 can parse the documents 104 to extract the atomic units. Each parser 112 can correspond to one or more types of modalities of the documents 104 and/or atomic unit types. The parsers 112 can perform preprocessing of documents 104, such as to process content of the documents 104, according to at least one type of modality of the document 104. In some implementations, the parsers 112 include at least one language model or embedding model, such as to generate tokens and/or vectors to represent (e.g., embed, encode) data of documents 104. In some implementations, each parser 112 can implement normalization or segmentation rules tailored to a specific modality type to prepare document content for atomic decomposition. For example, a parser 112 for textual input can divide sentences into token elements, a parser 112 for image input can detect pixels or region boundaries, and a parser 112 for audio input can divide waveform data into consecutive samples. In an example, a parser 112 applied to image-based text can use optical character recognition to identify character regions and associate coordinate metadata with extracted character tokens. Each parser 112 can provide the processed content to the atomic unit generator 108 for further transformation into atomic units (e.g., which the atomic unit generator 108 can represent in tables, e.g., records 120, of the database 116). In some implementations, one or more parsers 112 includes an optical character recognition (OCR) component. In some implementations, the atomic unit generator 108 includes one or more parsers 112.

The system 100 can route (e.g., transmit, direct) documents 104 and/or portions of documents 104, according to the modalit(ies) of the documents 104, to the corresponding parser 112 for the modalit(ies), such as to execute tokenization or segmentation functions. The system 100 can identify the corresponding parser 112 for each document 104 based on a detected modality of the document 104. For example, the system 100 can access metadata fields embedded in the documents 104 to identify an associated modality such as text, image, video, speech, or audio. For example, where the metadata specifies a text-based format, the system 100 can select the corresponding parser 112 that performs tokenization and sentence segmentation. Where the metadata specifies an image modality, the parser 112 can apply segmentation operations that determine pixel groupings or object boundaries for subsequent atomic processing. Each parser 112 can receive documents 104 through an automated routing process executed prior to atomic unit generation.

Referring further to FIG. 1, the atomic unit generator 108 can receive the output from one or more parsers 112, and can generate atomic representations of the output for relational storage. In some implementations, the atomic unit generator 108 can operate as a bridge between raw parsed content and structured relational data, creating a standardized representation compatible with relational database operations. The atomic unit generator 108 can interpret the tokenized or segmented output from modality-specific parsers and generate uniform data structures that encode both the content and contextual metadata of each atomic element.

For example, the atomic unit generator 108 can assign a unique atomic identifier (e.g., atomic unit ID 124) to each atomic unit (e.g. and without limitation, token, pixel, or audio sample), and can associate the unique atomic identifier with one or more attributes of the atomic unit, such as content or metadata of the atomic unit, including positional data, confidence metrics, and/or learned embedding vectors. These associations can allow for consistent and reproducible access to atomic data across retrieval sessions. The atomic unit generator 108 can further aggregate or normalize parser-generated attributes such as bounding box coordinates or timestamp values so that they can be stored as first-class relational attributes. The atomic unit generator 108 can execute iterative or streaming transformation processes that continuously process sequential segments of incoming data into atomic records, which can ensure that all relationally addressable elements are generated and captured in real time for storage or retrieval.

Referring further to FIG. 1, the system 100 can include a database 116. The database 116 can be a relational database and/or storage environment, which can maintain a corpus of atomic units. The system 100 can update the database 116 to represent atomic units generated by the atomic unit generator 108, e.g., as extracted from documents 104. In some implementations, the system 100 can use the database 116 as a foundational storage layer that supports query execution, relational joins, and/or aggregations over multimodal atomic data.

The system 100 can update the database 116 to include one or more records 120. The database 116 can include a table that indicates the records 120. Each record 120 can represent a corresponding atomic unit. In some implementations, the system 100 structures the database 116 and/or the records 120 to represent atomic units represented as rows in one or more interlinked tables. The database 116 can store a record 120 for each atomic unit extracted from the documents 104. Each record 120 can represent a granular relational entry corresponding to a single atomic unit generated from one of the documents 104. These records can act as the fundamental data blocks that capture all contextual and value-based information necessary for retrieval, enrichment, and recomposition of document fragments. In some implementations, each record 120 can include fields for the atomic unit ID 124, atomic unit content 128, and atomic unit attributes 132, such as to form an integrated schema that maintains direct relationships between a unit's identity, content, and metadata. For example, a record 120 may include a token from text with its unique ID, text string, and corresponding location data such as a character offset or bounding coordinates. These associations can also include document references that maintain a persistent link to the original unstructured file or source of extraction. The records 120 can thus function as base tables for relational operations-supporting selections, filters, joins, and aggregations used in retrieval workflows. As described further herein, the system 100 can receive and/or execute queries to compute aggregate statistics, apply relevance scoring functions, or generate chunk-level composites directly from fields defined within these records, enabling flexible and consistent access to atomic-level data throughout retrieval pipelines.

For example, the system 100 can assign, to each record 120, an atomic unit identifier (ID) 124, which can be a unique identifier for the atomic unit corresponding to the record 120. The atomic unit ID 124 can be a primary key for relational access. The atomic unit ID 124 can uniquely identify each atomic unit stored in the relational table and maintain referential integrity across all related data tables in the corpus. The system 100 can generate the atomic unit ID 124 can be generated using deterministic rules such as a composite of the document identifier, modality type, and intra-document offset, ensuring reproducible indexing across document updates. The system 100 can use the atomic unit ID 124 as a primary key used to join atomic unit records to metadata or chunk mappings and can facilitate relational operations that reconstruct semantic or structural groupings. For example, a paragraph or image region can be dynamically created by joining multiple atomic unit IDs 124 under a single chunk identifier (e.g., chunk ID 152). The atomic unit IDs 124 can provide consistency for cross-modal referencing; for example, a text token and an image region derived from the same page may be stored separately yet linked through the document identifiers to the document 104 of the page. Through these relationships, the atomic unit ID 124 enables traceability from high-level retrieval outputs back to the precise atomic elements that constitute them, which can support explainable and reproducible retrieval across modalities.

The system 100 can store, in each record 120, the corresponding data of the atomic unit as atomic unit content 128. The atomic unit content 128 can correspond to the extracted value of each atomic unit obtained from the documents 104, and can be used as the core payload for information retrieval. For example and without limitation, the system 100 can store text tokens, image pixels, and/or audio samples as the atomic unit content 128 (e.g., depending on the atomic unit type). Depending on modality, this content can represent a character sequence, a pixel intensity, or an audio waveform sample. In some implementations, the atomic unit content 128 can be stored as a normalized or tokenized value that allows semantic or numeric operations across units of different types. For text modalities, atomic unit content 128 can include tokens that are stored as strings or encoded representations for embedding or keyword-based processing. For image modalities, atomic unit content 128 may correspond to RGB or grayscale pixel values, while for audio modalities it may represent waveform samples or extracted spectral coefficients. These content fields can be fully queryable, enabling filtering or aggregation directly on the raw value while preserving associations with metadata. The system can join atomic unit content 128 with atomic unit attributes 132 to generate enriched outputs combining raw data and contextual descriptors, which allows retrieval processes to reconstruct text spans, image regions, or acoustic frames that satisfy specified relational criteria.

The system 100 can store, in each record 120, attributes of the atomic unit as atomic unit attributes 132. The attributes can include, for example and without limitation, an identifier of the document 104 from which the atomic unit was extracted, an indication of the atomic unit type of the atomic unit, positional attributes such as a relative or absolute location of the data (e.g., a position index, such as an ordinal position of the text in the document 104; pixel coordinates; time stamps of audio samples or image frames in video), confidence values associated with the parsing by parsers 112, such as OCR parsing scores; relevance scores; embedding vectors; similarity metrics; metadata extracted from the document 104; or various combinations thereof. For example, the atomic unit attributes 132 can include metadata and descriptive characteristics associated with each atomic unit, encompassing spatial, temporal, semantic, and confidence-related information. Using these attributes the system 100 can transform atomic content into richly annotated data elements, which can allow for advanced relational queries and contextual filtering. In some implementations, the atomic unit attributes 132 can include positional data such as coordinates or offsets within the original document, timestamps for audio or video frames, and derived values such as embedding vectors, semantic categories, or OCR confidence scores.

In some implementations, by storing metadata at the atomic level, the system 100 can allow for lossless preservation of spatial and structural details that can later be aggregated at higher levels. For example, the system 100 can execute retrieval queries to filter by bounding box coordinates, or can compute the mean semantic similarity of textual atoms within a given section. The atomic unit attributes 132 can also include both directly extracted and externally enriched data, allowing dynamic integration of additional information sources such as annotations, classifications, or relevance scores. This design allows the atomic unit attributes 132 to function as first-class fields in relational queries, enabling filtering, grouping, and ranking operations that combine content-based and metadata-based reasoning in a unified framework.

This structure of the database 116 and/or records 120 can allow for deterministic referencing and efficient reconstruction of higher-level document components. For example, the database 116 can include structured tables linking each atomic unit ID 124 with corresponding atomic unit content 128 and atomic unit attributes 132, forming extensible schemas capable of accommodating text, image, or audio-based information. The database 116 may maintain indexed columns on common attributes such as positional data, temporal identifiers, or semantic vectors to accelerate query performance. By leveraging these indexes, the system can efficiently perform complex relational queries such as grouping, joining, or aggregating atomic units to form higher-order chunks (e.g., chunks 144 as described further herein), such as pages, paragraphs, or regions of an image. Thus, the database 116 can serve as a comprehensive and modality-agnostic foundation for structured retrieval operations. Table 1 below provides examples of records 120 representing atomic units:

| | | Atomic Unit | Atomic Unit | Atomic Unit | Atomic Unit |
|---|---|---|---|---|---|
| Atomic Unit | Document | Content | Attributes | Attributes | Attributes |
| ID (124) | ID (132) | (128) | (132) | (132) | (132) |
| 101 | D01 | token text: | position | confidence | |
| | | diabetes | index: 15 | score: 0.98 | |
| 205 | P12 | OCR token | bounding | page | confidence |
| | | text: aspirin | box: {40, | number: 3 | score: 0.97 |
| | | | 120, 50, 15} | | |
| 302 | IMG2 | x coordinate: | y coordinate: | color values | region: R09 |
| | | 35 | 72 | (RGB): 128, | |
| | | | | 64, 120 | |
| 409 | AUD5 | timestamp: | sample | sample | |
| | | 3.54 s | amplitude: | frequency: | |
| | | | 0.047 | 315 Hz | |

Referring further to FIG. 1, the system 100 can include a selector 136. The selector 136 can select groups of atomic units, such as chunks 144 of atomic units, in response to any of various trigger conditions. For example, the selector 136 can select groups of atomic units in response to one or more requests 140 as described herein. The selector 136 can select groups based on scheduled or dynamic processes. The selector 136 can define the groupings dynamically, such as in response to the requests 140 (e.g., rather than the groups being defined based on and/or only on predefined indexing or chunking).

The selector 136 can function as a query execution component that applies relational expressions to perform filtering, grouping, or joining operations over atomic data maintained in the database 116. In some implementations, the selector 136 can evaluate relational expressions that reference atomic attributes 132 to determine which atomic units satisfy one or more conditions derived from query parameters. For example, the selector 136 can execute a query defined by a user or a system process in response to a request 140, can apply predicate logic to atomic unit attributes 132, and can return corresponding records 120 satisfying those conditions.

In some implementations, the selector 136 includes or is coupled with at least one application programming interface (API), which can allow for functions or methods to be defined for configuration of and/or processing of requests 140. For example, the selector 136 can include methods for retrieving data from the database 116 including one or more of a chunk method, an enrich method, a filter method, and a select method. The selector 136 can access, in response to the chunk method, an existing collection of chunks 144 by name, or can generate new chunks 144 (e.g., via an expression). From the resulting chunks object, the enrich method can be used (e.g., by the selector 136) to persist new attributes to chunks 144. The filter method can remove chunks 144 based on attributes or expressions. The select method can assign chunk and atom metadata into a table for downstream use. The request 140 can be one or more requests in which any of various such methods of the selector 136 can be chained to construct complex data transformations.

The selector 136 (e.g., the API of the selector 136) can receive expressions that define functions or operations to compute. The expressions can be associated with the API. The expressions can include attribute expressions that represent chunk-level attributes (which, for example, the selector 136 can compute and can store as chunk attributes 148). The expressions can include chunk expressions, which can define chunking strategies over the atomic data units, such as sliding windows. The expressions can include chunk filter expressions, such as to define chunk filtering approaches such as top K or minimum thresholds that can be applied to existing chunk attributes 148 or for determination of chunk attributes 148. The expressions can be user-definable.

Referring further to FIG. 1, the system 100 can receive one or more requests 140 for data, e.g., atomic units, from the documents 104. The selector 136 can generate a response to the request 140, such as to output records 120 or data of records 120, according to one or more criteria indicated by the request 140. For example, the requests 140 can represent incoming retrieval expressions that define selection or grouping instructions for accessing atomic unit data within the database 116. In some implementations, each request 140 can specify retrieval parameters such as a collection name, atomic attribute filters, top-K constraints, or threshold values for one or more relevance attributes. For example, a request 140 can include parameters indicating search terms or embedding-based similarity conditions that identify atomic units to obtain or to combine into chunks 144. Each request 140 can serve as a query object containing composable expressions representing content selection logic, enrichment logic, or scoring stages. In some implementations, the requests 140 can originate from an application interface or an external system utilizing the corpus query application programming interface to initiate relational retrieval.

Referring further to FIG. 1, the selector 136 can generate a chunk 144 of atomic units. The selector 136 can generate the chunk 144 to be a data object. The chunk 144 can be a group, e.g., a collection, of atomic units, such as meaningful units to retrieve or reference (e.g., in response to a given request 140). For example, the selector 136 can generate the chunk 144 to include selected atomic units to meet attribute filters or aggregation criteria expressed by a retrieval request 140. As an example, the selector 136 can apply relational HAVING clauses to construct a chunk corresponding to a phrase, sentence, or paragraph, or can apply scalar and vector aggregation functions to compute one or more chunk-level results. The selector 136 can retrieve partial groupings or compound aggregations of atomic unit IDs 124, and can assign results to alias tables for use in subsequent query stages. In some implementations, the selector 136 can evaluate sequential queries or pipeline operations forming multi-stage retrieval workflows that allow distinct ranking expressions or attribute filters at successive retrieval stages.

The chunk 144 can represent a relational grouping or dynamically created view of atomic unit records, which can collectively form a retrieval unit for the response to a request 140. In some implementations, the chunk 144 can represent any subset of atomic units defined by expressions specifying spatial, temporal, or semantic boundaries. For example, the chunk 144 can correspond to a contiguous group of text tokens within a paragraph, a region of pixels in an image, or a selection of audio samples associated with a time interval. The selector 136 can assign a chunk identifier 152 to the chunk 144 as a unique identifier for the chunk 144.

The system 100 can generate each chunk 144 on demand, such as by execution of a query interpreted by the selector 136. The selector 136 can represent the chunk 144 as a relational table or view mapping the chunk identifier 152 to a set of atomic unit identifiers 156 and one or more chunk-level attributes 148. In some implementations, the chunk 144 can be a dynamically generated result set rather than a persistently indexed entity within the corpus. For example, a relational join expression may compute grouping keys based on text span boundaries or bounding box coordinates and produce a corresponding chunk 144 for downstream use in ranking or display operations. Each chunk 144 can provide the basis for context aggregation, cross-modal enrichment, or temporal correlation of atomic-level data during retrieval.

For example, the chunk 144 can include or be represented as including one or more chunk attributes 148. The chunk attributes 148 can include chunk metadata. The chunk attributes can include relevance scores, embeddings, text representations, or bounding boxes, for example. The chunk attributes 148 can capture aggregated or derived metadata representing properties associated with each chunk 144. In some implementations, the chunk attributes 148 can include precomputed or dynamically computed values produced through aggregation over one or more atomic attribute fields. For example, chunk attributes 148 can include mean or maximum relevance scores, combined embedding vectors, average OCR confidence scores, or bounding box aggregates derived from constituent atomic units.

The selector 136 can access or compute chunk attributes 148 to rank, filter, or recombine chunks within a retrieval query. The selector 136 can maintain the chunk attributes 148 in a relational table that stores the chunk identifier 152 as a primary key and associates each aggregated attribute value with the corresponding chunk identifier through join operations. In some implementations, the selector 136 can perform join operations across the relational table and one or more auxiliary tables that contain atomic unit identifiers or intermediate aggregation results. For example, the selector 136 can execute a join between a chunk attribute table and an atomic unit table to compute aggregated fields such as mean embedding vector values, cumulative bounding box regions, or combined relevance scores associated with each chunk identifier 152.

The selector 136 can update or regenerate the chunk attributes 148 during query evaluation to reflect relational aggregations that derive from atomic-level attributes 132, allowing each chunk identifier 152 to reference a coherent set of computed attribute values accessible for downstream selection or ranking operations. For example, calculation of a combined similarity metric from multimodal inputs can generate a chunk attribute representing fused relevance between text and image modalities. Derived chunk attributes 148 can be expressed as relational projections or functions within query definitions that extend or refine retrieval output structure over atomic-level records.

The chunk identifier 152 can serve as a unique key that distinguishes each chunk 144 within the corpus and facilitates relational joins linking chunk-level data to underlying atomic unit records. In some implementations, the chunk identifier 152 can be generated by the selector 136 upon creation of a new chunk view or can correspond to an existing entry within the database 116. For example, a newly computed paragraph-level chunk may be assigned a chunk identifier 152 that links to atomic unit identifiers 156 in a mapping table maintained within the database 116. The chunk identifier 152 can identify a record within a chunk attribute table while maintaining a one-to-many relationship to the atomic unit identifiers referenced from the atomic unit table. In some implementations, relational integrity between the chunk identifier 152 and the atomic unit identifiers 156 can be maintained through foreign key constraints enforced within the schema. For example, a join operation associating a chunk identifier 152 with its atomic unit identifiers 156 can reconstruct the composition of a multi-modal retrieval chunk derived from text, image, or audio atomic units in response to a retrieval request 140.

The atomic unit identifiers 156 can be or correspond to the atomic unit IDs can represent relational references linking atomic units to corresponding chunks 144 and can define the membership of atomic data records used in retrieval. In some implementations, the unit identifiers 156 can associate atomic unit identifiers 124 drawn from text, image, or audio modalities with a specific chunk identifier 152 defining a retrieval grouping. For example, a chunk 144 representing a paragraph may link ten token-based unit identifiers 156 and two image-region identifiers within one mapping table that establishes the complete multimodal context. Each record in the mapping table can include a chunk identifier 152 and one or more atomic unit identifiers 156, which can allow for bidirectional queries from chunk to atomic records or vice versa. In some implementations, the system 100 can access, based on retrieval queries represented by the requests 140, the mapping table to perform join operations that reconstitute full chunk content and attributes for query results. For example, the selector 136 can combine the atomic content associated with unit identifiers 156 to generate reconstructed composite views of text segments, image regions, or audio clips for delivery in response to a retrieval request 140.

As an example, the selector 136 can receive a request 140 that includes the following query with respect to processing document OCR data that includes text and spatial coordinates:

(corpus.chunk(“token”)
.filter(TopK(“confidence”, 10))
.select(text=SimpleStringify( ), bbox=AtomData(“bbox”)))

The selector 136 can perform multi-stage retrieval. For example, the selector 136 can perform a first selection of atomic units and/or chunks 144 according to a first request 140, and can perform a second selection of atomic units and/or chunks 144 according to a second request 140. As an example, a series of sequential requests can specify a first scoring stage using BM25 relevance functions and a second scoring stage for semantic re-ranking using embedding similarity. Each request 140 can be evaluated by the selector 136 to produce or modify the composition of one or more chunks 144 within the database 116 in response to specific data retrieval requirements. As in the following example, the system 100 can perform a first retrieval (e.g., using fast BM25), and can perform a second retrieval by re-ranking candidates with semantic similarity:

(corpus.chunk(FixedSizeChunk(“paragraph”, 100))
.enrich(text=SimpleStringify( )) # Pre-store text for convenience
# Initial retrieval using fast BM25
.filter(TopK(BM25(attr=“text”, query=“my query”), 1000))
# Re-rank top candidates with semantic similarity
.filter(TopK(SemanticSimilarity(attr=“text”, query=“my query”), 10))
.select(text=SimpleStringify( )))

In response to the request 140, the selector 136 can retrieve chunks 144 of atomic units (e.g., based on records 120) that correspond to the “token,” can filter the retrieved chunks 144 for the top ten chunks 144 based on confidence (e.g., with respect to the token), and can output chunk 144 and atomic unit data and/or metadata according to text and bounding box information indicating spatial coordinates to select. As compared to document retrieval systems that treat documents as monolithic objects and/or rely on index-time chunking, the system 100 can thus support rich, multi-granular metadata as first-class attributes that can be queried alongside the document 104.

As noted above, the system 100 can allow for dynamic chunking and/or view-based retrieval. For example, the system 100 can extract atomic units, can store the extracted atomic units in records 120, and can retrieve data from records 120 upon receiving requests 140, which can avoid the need for upfront chunk persistence or re-indexing (including, for example, re-indexing and/or re-chunking each time a distinct query is received). The following example indicates how the system 100 can form chunks 144 from atomic units from a document 104, can enrich the chunks 144 by forming embeddings of the text of the chunks 144, can filter the chunks 144 according to similarity between the embeddings and a query, and can generate an output according to the filtered chunks 144:

(corpus.chunk(FixedSizeChunk(“document”, 100))
.enrich(embedding=BertEmbedding(SimpleStringify( ))) # Embed text
.filter(TopK(BertSimilarity(“embedding”, query=“my query”), k=10))
.select(“id”))

Table 2 below provides examples of greater retrieval speed as achieved by the system 100, such as for end-to-end retrieval speed including indexing.

| | | | TREC-COVID (171000 | |
|---|---|---|---|---|
| | NFCorpus (3600 documents) | | documents) | |
| | Pyserini | System 100 | Pyserini | System 100 |
| Max time | 5.24 | 1.16 | 12.74 | 17.12 |
| (seconds) | | | | |
| Mean time | 4.54 | 1.09 | 12.16 | 16.46 |
| (seconds) | | | | |
| Min time | 4.17 | 1.05 | 11.47 | 15.80 |
| (seconds) | | | | |

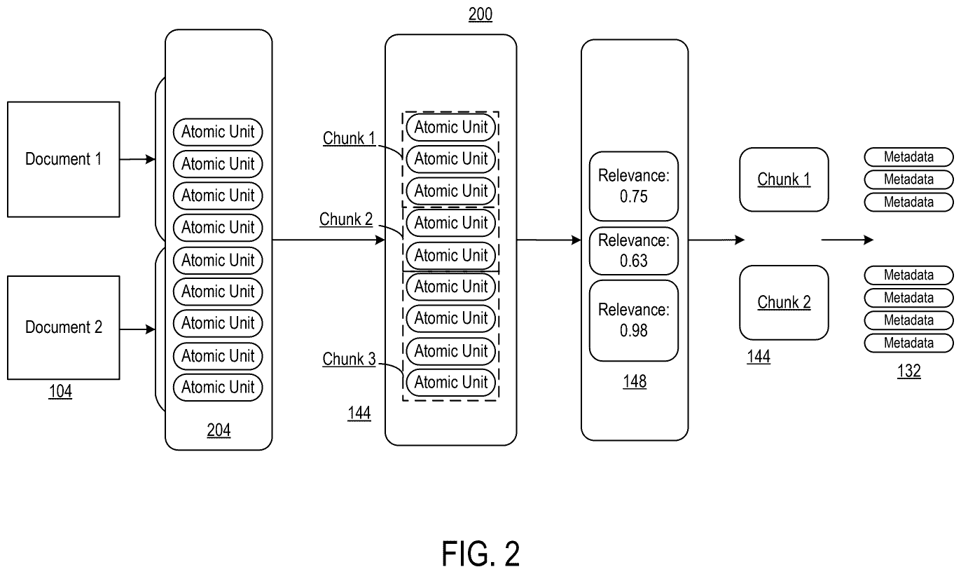

FIG. 2 depicts an example of a process 200 of data retrieval that the system 100 can perform. For example, the system 100 can perform the process 200 to generate atomic units and/or in response to a request 140 for data from documents 104.

For example, the system 100 can cause parsing of a first document 104 and a second document 104 to extract a plurality of atomic units, such as words, tokens, pixels, or audio samples, for example and without limitation. The atomic units can form a corpus 204; for example, the system 100 can maintain the corpus 204 in the database 116. The system 100 can define a first chunk 144, a second chunk 144, and a third chunk 144 from the atomic units, each chunk 144 corresponding to associated atomic units.

As depicted in FIG. 2, the system 100 can determine (e.g., based on one or more criteria indicated by the request 140) chunk attributes 148, such as relevance scores for atomic units of the chunks with respect to the request 140. The system 100 can determine a respective chunk attribute 148 for each chunk 144, which can be based on atomic unit attributes 132 of the atomic units of the respective chunks 144.

The system 100 can filter the chunks 144 according to the chunk attributes 148, such as to select the first and third chunks 144 (e.g., based on a threshold relevance score, or a request to select the top two chunks 144). The system 100 can provide output that includes data and/or metadata of the atomic units of the selected chunks 144, such as requested atomic unit attributes 132, such as text contents, token location information, pixel values, for example and without limitation; such data can be accessed regardless of how it was retrieved.

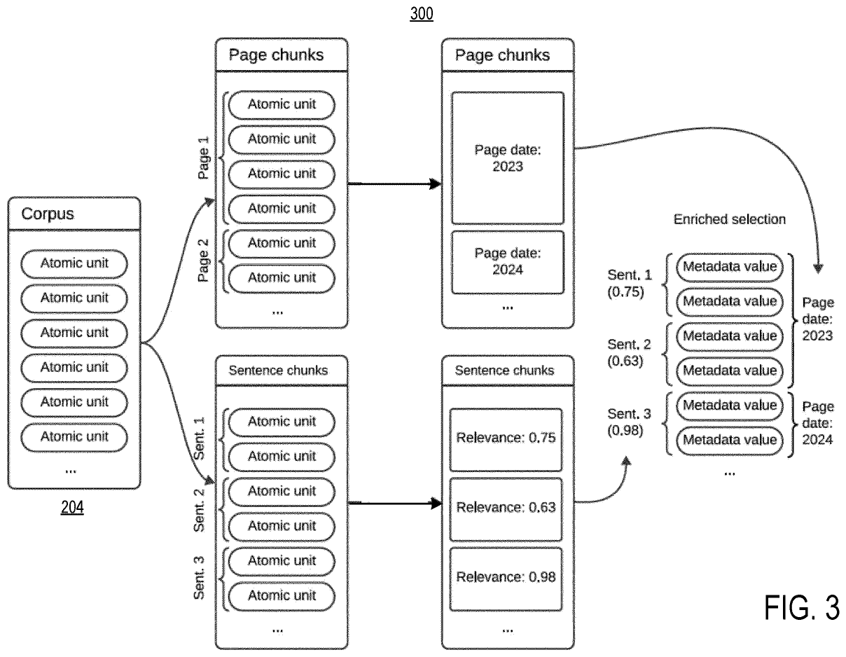

FIG. 3 depicts an example of a process 300 that the system 100 can perform. For example, in the process 300, the system 100 can define multiple types of chunks 144 for a given corpus 204, rather than requiring re-indexing and/or multiple sets of chunks to be stored.

For example, the system 100 can generate each of page chunks 144 (e.g., chunks 144 corresponding to atomic units that make up respective pages of a given document 104) and sentence chunks 144 (e.g., chunks 144 corresponding to atomic units that make up respective sentences of a given document 104) based on the atomic units of the corpus 204. The system 100 can determine, for each of the page chunks 144, chunk attributes 148 such as the page date of the respective page one (2023) and page two (2024). The system 100 can determine, for each of the sentence chunks 144, chunk attributes 148 such as relevance scores of each of the respective sentences with respect to a query, for example. As depicted in FIG. 3, the system 100 can generate an enriched output that includes each of the page-level chunk attributes 148 of page dates as well as the sentence-level chunk attributes 148 of sentence-level relevance scores.

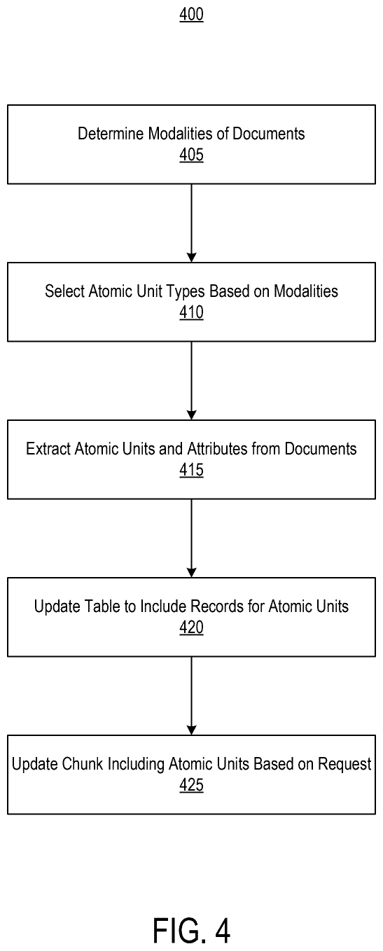

Referring now to FIG. 4, illustrated is a method 400 of atomized relational retrieval, in accordance with one or more implementations. The method 400 can be executed, performed, or otherwise carried out by any of the computing systems or devices described herein. In brief overview of the method 400, the method 400 can include determining modalities of documents 405, selecting atomic unit types based on modalities 410, extracting atomic units and attributes from documents 415, updating a table to include records for atomic units 420, and updating a chunk including atomic units based on a request 425.

At 405, the method 400 can include determining modalities of documents. The modalities can be determined subsequent to ingestion of the documents, including in continuous or batch processing of documents or portions of documents. The system can determine a modality type for each document among a plurality of documents to establish the appropriate processing pipeline. In some implementations, the system can classify documents as text, image, audio, or other modalities based on embedded metadata, format signatures, or document headers. The determination can occur as an initial stage preceding atomic unit extraction so that subsequent parsing operations are aligned with the detected modality type. In some implementations, the system can perform this determination immediately after receiving the documents from a file ingestion interface or a corpus loader component. In some implementations, multiple modalities are determined for any given document, e.g., based at least on the given document having data of multiple modalities, such as both text and image content.

At 410, the method 400 can include selecting atomic unit types (e.g., for data of the documents) based on the determined modalities. For example, the system can select an atomic unit type for each document according to the determined modality for each document. In some implementations, textual documents can be determined to have atomic unit types of text or tokens, image documents can be determined to have atomic unit types of pixels or image regions, and audio documents can be determined to have an atomic unit type of audio samples. For example, a mapping function can associate identified modality indicators with corresponding parsers or atomic unit generators that perform segmentation or feature extraction. The selection can occur after modality identification and before extraction and table updates, providing consistency across downstream relational operations. In some implementations, the system can reference a stored configuration that links text modality with a tokenizer, image modality with a pixel sampler, and audio modality with a waveform segmenter, ensuring alignment between parsing logic and data modality.

At 415, the method 400 can include extracting atomic units and attributes of the atomic units from the documents. For example, the system can parse unstructured data of each document according to its selected atomic unit type to derive atomic units and associated attributes. In some implementations, each extracted unit can include or be associated with contextual metadata such as positional coordinates, timestamps, or confidence values generated by the modality-specific parser. The extraction can occur after completion of atomic unit type selection and before relational table updates, such as to preserve ordered data flow across pipeline stages. In some implementations, parser output pipelines can compute embeddings, coordinate mappings, or segmentation indices as atomic attributes prior to insertion into the relational corpus.

At 420, the method 400 can include updating a table to include records for atomic units. For example, the system can update a relational table and/or database to insert a record for each extracted atomic unit. In some implementations, each record can store a unique identifier for the atomic unit, a document identifier for the document from which the atomic unit is extracted, content (e.g., data) of the atomic unit, and one or more attributes of the atomic unit, such as one or more attributes derived from the extraction process. For example, when processing a PDF, a token extracted from a page can be recorded as a new row including a token ID, textual content, and positional coordinates that identify its position in the source document. The table update can occur after atomic unit extraction and before any chunk generation or retrieval queries. In some implementations, the table update can be implemented using relational insertion operations or batch appends to a corpus-wide atomic table to maintain a persistent mapping between documents, atomic identifiers, and extracted attribute fields.

At 425, the method 400 can include generating and/or updating a chunk to selected atomic units, such as based on a request or query. For example, the system can output, in response to a retrieval request referencing one or more atomic units, at least one record corresponding to a dynamically defined chunk. In some implementations, the system can generate the chunk definition using one or more selection criteria such as relevance, position, or embedding similarity specified in the request. For example, a query can specify selection of tokens exceeding a confidence threshold or combined page regions containing related features across modalities. The chunk update can occur after the atomic unit table is populated and can be triggered by execution of a retrieval query requiring multi-resolution or multi-stage output. In some implementations, the system can update the chunk by defining a relational view or a temporary table that references atomic unit identifiers and corresponding chunk-level metadata such as bounding-box aggregates, semantic embeddings, or calculated relevance values.

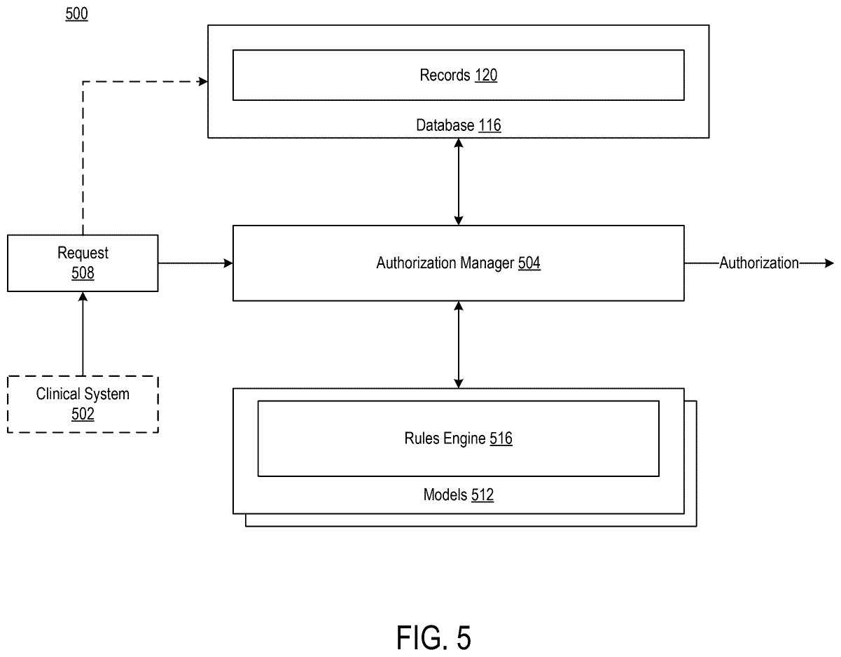

Referring now to FIG. 5, illustrated is a block diagram of an example of a system 500, e.g., an authorization system 500, for processing clinical requests, in accordance with one or more implementations. The system 500 can be used to process clinical requests for authorization of clinical actions. For example, the system 500 can facilitate at least some automated electronic processing of requests for authorization of clinical actions.

In some implementations, the system 500 includes or is coupled with one or more components of the system 100, such as the atomic unit generator 108, the database 116, and/or the selector 136. For example, the system 500 can execute or provide instructions to one or more components of the system 100 to execute operations for extracting atomic units from documents 104. For example, the system 100 can manage the database 116, such as to establish, modify, or update the database 116. In some implementations, the system 500 can manage operations of the database 116 associated with documents 104; for example, the system 500 can cause the atomic unit generator 108 to extract atomic units from documents 104 that may represent clinical information, and can update the database 116 to include records 120 that represent the atomic units, the atomic unit content 128 of the atomic units, and the atomic unit attributes 132 of the atomic units.

As noted above, at least some of the documents 104 can represent clinical information. For example, the documents 104 can include or represent any of various forms of clinical and medical data such as electronic health records, diagnostic imaging reports, or laboratory test results, among others. In some implementations, the documents 104 can include scanned facsimile (fax) documents or portable document format (PDF) files that represent handwritten clinical notes, referral forms, or prior authorization requests. For example, a document 104 can include a structured electronic chart describing patient demographics and encounter summaries, a digital radiology study that includes both image pixel data and DICOM (Digital Imaging and Communications in Medicine) metadata, and/or a claims record specifying procedural and diagnostic codes associated with a submitted service request. In some implementations, the documents 104 can include guideline or policy references outlining recommended criteria for approving clinical actions such as prescriptions, imaging tests, or surgical procedures, providing direct contextual information for processing an authorization request.

Referring further to FIG. 5, the system 500 can include an authorization manager 504. The authorization manager 504 can perform operations for automating authorization of clinical actions. For example, the authorization manager 504 can receive a request 508 for authorization of a clinical action. The request 508 can include data fields specifying parameters associated with the clinical action to be authorized. In some implementations, the request 508 can include at least one of an identifier of a patient or subject for whom the clinical action is to be performed, a classification of the clinical action to be carried out, a location at which the clinical action is to occur, or a designation of a clinician type or specialty requested to perform the clinical action. For example, the request 508 can specify a patient identifier, a treatment or diagnostic procedure code, a facility identifier corresponding to a hospital or outpatient site, and a provider classification such as radiologist, cardiologist, or orthopedic surgeon, among others. The request 508 may be received as part of or to initiate an intake process for a patient, or may correspond to a clinical action for which intake has previously been performed for the patient. The request 508 can include associated documents 104. The request 508 can be received by way of any of various electronic communication channels.

In some implementations, the clinical action includes or represents a type of the clinical action. For example, the type can indicate a category or class of the clinical action, such as a category of procedure to be performed. In some implementations, the system 500 uses an identifier of the clinical action (e.g., a text label or numeric identifier) as the type of the clinical action. In some implementations, the request 508 indicates each of the identifier of the clinical action and the type of the clinical action.

In some implementations, the system 500 includes or is coupled with a clinical system 502. For example, the system 500 can receive the request 508 from the clinical system 502. The clinical system 502 can be maintained by an entity separate from or integrated with an entity that maintains the system 500 and/or the system 100. The clinical system 502 can include or be coupled with, for example, a clinical management system or an electronic medical record processing system. In some implementations, the clinical system 502 is maintained by a provider entity, such as a provider seeking authorization from the system 500 to perform the clinical action. The system 500 can provide one or more interfaces, such as APIs or portal interfaces, to facilitate communication between the system 500 and the clinical system 502.

Referring further to FIG. 5, the system 500 can include one or more models 512. The models 512 can be or include any of various processors, hardware, software, databases, algorithms, functions, modules, neural networks, machine learning models, heuristics, policies, rules, or various combinations thereof to perform operations on data in the system 500, including but not limited to intake, document parsing, data extraction, validation, decision, and/or review operations. One or more models 512 can be structured to perform specific tasks, and may include identifiers indicating the respective tasks, which can allow the system 500 to route operations in an authorization process to corresponding models 512.

In some implementations, one or more models 512 includes a corresponding rules engine 516, such as a clinical rules engine 516. The rules engine 516 can identify one or more rules to process data regarding the clinical action, such as to provide a framework for automating decisions for authorization of the clinical action, such as to approve or deny the request for authorization of the clinical action. For example, based on one or more of the type of the clinical action or data available for evaluation of the authorization, the rules engine 516 can select one or more rules. The models 512 can include clinical evidence models structured to evaluate clinical evidence against the one or more rules.

The one or more rules can include logic or conditions that the rules engine 516 can evaluate according to received inputs, such as data that the authorization manager 504 retrieves from the database 116. The rules engine 516 can evaluate rules in any of various orders, including sequentially or in parallel, or using voting methods. In some implementations, the models 512 and/or the rules engine 516 can include one or more machine learning models to facilitate the data processing and/or evaluation of rules. The rules can include deterministic rules and/or probabilistic rules.

In some implementations, the authorization manager 504 identifies a rules engine 516 and/or one or more rules that correspond to the clinical action, such as to correspond to the type of the clinical action. For example, the authorization manager 504 can select rules that address authorization of the clinical action. The authorization manager 504 can select rules that correspond to information in the request 508.

Referring further to FIG. 5, the authorization manager 504 can identify, submit, and/or generate a query to the database 116 to retrieve data, e.g., a chunk, from the database 116. The authorization manager 504 can generate the query based on one or more of the request 508, the identifier of the rules engine 516, or the selected one or more rules. The authorization manager 504 can generate the query to include an identifier of the type of the clinical action and/or a type of chunk relevant to the type of the clinical action. For example, the authorization manager 504 can generate the query to indicate document structures or sections, data types, or data corresponding to chunk attributes 148. In some implementations, the authorization manager 504 generates the query to select for one or more guideline or protocol documents related to the type of clinical action, such as guidelines that the rules engine 516 can use to evaluate data from the database 116.

In some implementations, the authorization manager 504 generates the query to include one or more filters, which can be used to select chunks 144 and/or atomic units relevant to the one or more rules and/or the request 508. For example, the authorization manager 504 can generate the query to include one or more filters corresponding to the functions, methods, and/or expressions of the selector 136, such as the chunk, enrich, filter, and/or select methods, the attribute expressions, and/or the chunk expressions. In some implementations, the authorization manager 504 (or the rules engine 516) determines at least one of the type of chunk or the one or more filters based on one or more expected inputs for evaluating the request 508, such as input data types or attributes or documents indicated by the one or more rules, or an identifier of the patient (e.g., to retrieve documents regarding the patient). The input data types or attributes can correspond, for example, to types of patient data, notes, lab results, provider recommendations, medications, previous or current clinical actions, treatments, or prescriptions, or various combinations thereof. The authorization manager 504 can generate the query to include one or more filters relevant to the type of the clinical action, such as to filter for document types relevant to the type of the clinical action. For example, the authorization manager 504 can generate the query to include at least one filter to select for medical records of a patient identified in the request 508.

The authorization manager 504 can input the query to the database 116 to retrieve, from the database 116, at least one of a group (e.g., chunk 144) of atomic units based on the query or data (e.g., values) of the group of atomic units. For example, the authorization manager 504 can use the query to retrieve a chunk 144 of atomic units that can correspond (or may be expected to correspond) to the type of the chunk and/or the one or more filters, such as to retrieve data relevant for evaluating the one or more rules. In some implementations, the authorization manager 504 identifies at least some of the one or more rules according to the retrieved group of atomic units, such as to add or remove rules that may be relevant once potential data that can be used to evaluate the rules is identified.

Referring further to FIG. 5, the authorization manager 504 can determine, by the rules engine 516 using the one or more rules, whether the data of the chunk 144 of atomic units resulting from the query satisfies the one or more rules. For example, the authorization manager 504 can use the rules engine 516 to identify data from the retrieved atomic units (e.g., content, metadata, and/or attributes) that corresponds to expected input(s) for each rule, and input the corresponding data into the rules to cause generation of an output for each rule. For example, the authorization manager 504 can provide the retrieved atomic unit data to the rules engine 516 for evaluation against the one or more rules identified for the type of clinical action. The rules engine 516 can execute each rule using the atomic unit attributes and metadata as input variables to determine whether specified conditions are satisfied. In some implementations, the rules engine 516 can apply threshold comparisons, pattern-matching logic, or relational predicates defined within the rules to generate the outputs, e.g., a determination result that indicates compliance with authorization criteria indicated by the one or more rules. The rules engine 516 can generate the output to include a confidence score associated with the evaluation, which may facilitate downstream review or auditing of the determination. The rules engine 516 can generate a plurality of initial outputs from at least a subset of the one or more rules, and apply one or more further rules or voting methods to the initial outputs to determine a final output.

Based on determining that the retrieved data satisfies the one or more rules, the authorization manager 504 can authorize the clinical action. For example, the authorization manager 504 can provide (e.g., output, transmit) a data structure or signal that includes an indication that the clinical action is authorized. In some implementations, the authorization manager 504 generates the indication to include supporting data for the decision to authorize the clinical action, such as the content of the atomic units inputted into the one or more rules, or corresponding documents (which the authorization manager 504 can retrieve using the document identifier of the atomic unit attributes 132). The authorization manager 504 can output the indication to a system that provided the request 508, such as to the clinical system 502.

In some implementations, the authorization manager 504 presents an indication of the (approval of or denial of) the authorization for confirmation by a user. For example, the authorization manager 504 can present the indication as a candidate determination that the group of atomic units resulting from the query satisfy the one or more rules. The authorization manager 504 can present, using a user interface, an indication of the candidate determination. The indication can include text describing the candidate determination, and can include or link to data used to evaluate the one or more rules to arrive at the candidate determination. The authorization manager 504 can receive, via the user interface, a confirmation of the candidate determination. The authorization manager 504 can authorize the clinical action based on the confirmation.

In some implementations, the authorization manager 504 includes or is coupled with at least one inferencing pipeline, such as one or more neural networks that provide a model inferencing pipeline. The authorization manager 504 can provide the retrieved data to the inferencing pipeline to cause the inferencing pipeline to process the retrieved data, such as to modify (e.g., increase, boost) one or more characteristics of the retrieved data, such as at least one of accuracy, precision, or recall of the retrieved data. The one or more characteristics can be boosted, for example, based on implicit or explicit measures of the characteristics for relevance of the retrieved data to the query and/or the one or more rules. In some implementations, the inferencing pipeline includes one or more machine learning models configured (e.g., trained, fine-tuned, updated, etc.) to generate feature representations of the data (and/or chunks 144), such as chunk content, metadata, or multimodal data. The inferencing pipeline can re-score relevance based on semantic similarity to the query. The inferencing pipeline can filter out chunks 144 (or data thereof) with mismatched contextual attributes. The inferencing pipeline can trigger additional queries, such as to identify additional chunks 144 related by semantic or metadata-based associations not present in the initial retrieval. By re-ranking, filtering, and expanding the candidate set, for example, the authorization manager 504 can the pipeline increases the likelihood that chunks 144 relevant to the clinical action are included and/or that irrelevant chunks are excluded prior to rule evaluation, which can improve the accuracy and efficiency of the authorization determination.

The authorization manager 504 can generate audit data regarding the determination. For example, the audit data can include content of at least one atomic unit of the group of atomic units, and can include a location of the content in a corresponding document of the plurality of documents from which the at least one atomic unit is extracted. In some implementations, the authorization manager 504 can associate the location data with identifiers of the documents 104 to provide a relational mapping between extracted atomic units and their source positions within the original clinical files. For example, the authorization manager 504 can reference positional attributes of atomic unit attributes 132, such as coordinate offsets or page indices, to specify the precise segment of a document 104 corresponding to the evaluated content used in the authorization process. The authorization manager 504 can generate the audit data such that each atomic piece of evidence used in the automatic approval (or denial) decision performed by the authorization manager 504 can be traceable back to the atomic source data, e.g., text and image source, of that evidence.

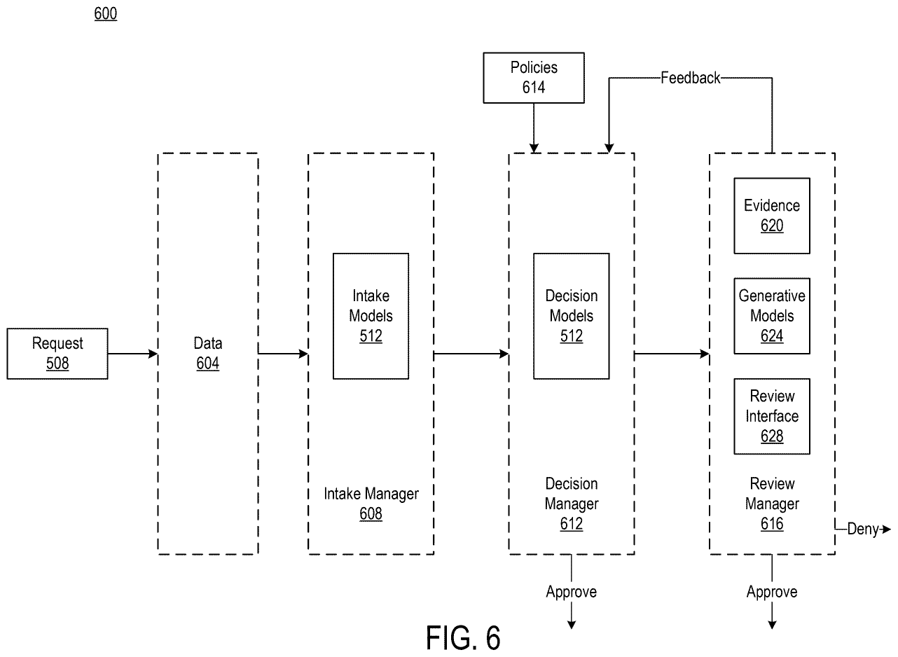

Referring now to FIG. 6, illustrated is a block diagram of an example workflow 600, such as a decision making workflow, in accordance with one or more implementations. The workflow 600 can be implemented by any of various systems or components described herein, including, for example, the authorization manager 504, the system 500, and/or the system 100.

As depicted in FIG. 6, the request 508 can be received. The request 508 can be parsed to identify data 604, such as case data and/or member data. For example, the data 604 can include any one or more attachments (e.g., via fax or electronic portal communication), metadata regarding the request 508, claims and/or authorization history data, or various combinations thereof. In some implementations, data from the request 508 is provided to the system 100 for atomic unit extraction.

Referring further to FIG. 6, the workflow 600 can include providing data 604 to an intake manager 608. The intake manager 608 (as well as decision manager 612 and review manager 616) can be layers of the system 500. The intake manager 608 can perform any of various intake operations such as storing or modifying the data 604. As depicted in FIG. 6, the intake manager 608 can be include one or more intake models 512. The intake models 512 can perform operations such as parsing attachments of the data 604, extracting case data from the data 604, or validating data 604. Any one or more of the intake models 512 (as well as decision models 512, the review manager 616, and/or components of the review manager 616) can include or be coupled with components of the systems 100, 500, such as to use the atomic unit generator 108 to extract atomic units from the request 508 or data 604, or to query the database 116 for relevant chunks of data for evaluation of the request 508. For example, the intake models 512 (e.g., directly and/or via extraction of atomic units) can parse clinical attachments to convert images, hand-written notes, and/or tables to structured segments. The intake models 512 can extract information from segmented documents to pre-populate an intake form. The intake models 512 can validate completeness and/or relevance of submissions.

The workflow 600 can include providing data 604 (e.g., subsequent to processing by the one or more intake models 512) to a decision manager 612. The decision manager 612 can perform at least a candidate determination of whether to authorize the clinical action indicated by the request 508. The decision manager 612 can be or include one or more decision models 512, which, as described with respect to models 512 and/or rules engines 516 with reference to FIG. 5, can evaluate one or more rules to determine whether to authorize the clinical action. The decision models 512 can evaluate and/or include rules to evaluate data such as clinical evidence or clinical assessments. In some implementations, the decision manager 612 can output an approval of the request 508 or a pending approval of the request 508 for further review. In some implementations, the decision models 512 (e.g., directly and/or via extraction of atomic units) can perform operations such as to extract clinical attributes to input to rules that can perform automated approval, or to estimate likelihood of the request being approved.

In some implementations, the decision manager 612 can access one or more policies 614 to evaluate the one or more rules. For example, the policies 614 can include the one or more rules, or be used as input for the one or more rules. The policies 614 can include, for example, guidelines on what clinical actions may be authorized given certain clinical data, evidence, or assessments.

The workflow 600 can include providing a review manager 616 one or more outputs from the decision manager 612. For example, The review manager 616 can operate to evaluate the decision output generated by the decision manager 612 using automated or semi-automated processes. The review manager 616 can apply one or more generative models 624, such as language models or vision-language models, to re-evaluate the decision output and generate a candidate explanation, summary, or alternative conclusion aligned with the decision criteria. In some implementations, the review manager 616 can compare the regenerated output against the decision output from the decision manager 612 to identify areas of inconsistency, missing context, or low-confidence determinations. For example, a generative model 624 can generate text describing clinical justification or visual annotations corresponding to supporting evidence detected in attachments, and the review manager 616 can associate the generated content with portions of the retrieved data. In some implementations, the review manager 616 can present a candidate output through the review interface 628 to a reviewer, who can confirm or modify the candidate output based on the displayed evidence 620. The review manager 616 can thereby facilitate confirmation, correction, or supplementation of the decision output prior to final authorization. The review manager 616 or components thereof can perform operations such as to rationalize extracted clinical information against policies 614, assist reviewers in locating missing evidence, generate review notes summaries, and/or prepopulate the decision outputs.

Referring further to FIG. 6, the workflow 600 can output an indication of the evaluation of the request 508, such as to approve or deny the request. In some implementations, the output of the review manager 616 is provided as feedback to the decision manager 612, and the decision manager 612 can update the determination according to the feedback. This can be used, for example, to update or optimize the decision models 512.

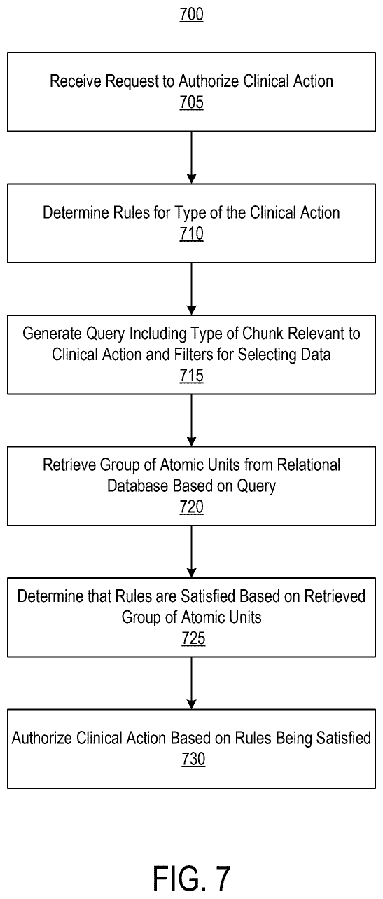

Referring now to FIG. 7, illustrated is a flow chart of a method 700 for authorizing a clinical action using relational retrieval of atomic units, in accordance with one or more implementations. The method 700 can be executed, performed, or otherwise carried out by any of the computing systems or devices described herein. In brief overview of the method 700, the method 700 can include receiving a request to authorize a clinical action 705, determining rules for a type of the clinical action 710, generating a query including a type of chunk relevant to the clinical action and filters for selecting data 715, retrieving a group of atomic units from a relational database based on the query 720, determining that the rules are satisfied based on the retrieved group of atomic units 725, and authorizing the clinical action based on the rules being satisfied 730.

At 705, the method can include receiving a request to authorize a clinical action. The request can be received from a clinical system, an intake application, a portal, or another computing system associated with a provider or payer network. In some implementations, the request can include identifying information such as a patient identifier, a clinician identifier, or an action code specifying the type of procedure to authorize. For example, the request can include structured data fields representing a treatment, diagnostic test, or referral request, along with associated metadata such as timestamps, facility identifiers, or document attachments describing clinical justification.

At 710, the method can include determining rules for a type of the clinical action. For example, a rules engine can identify one or more rules based on the classification or type of the clinical action indicated in the request. In some implementations, a stored mapping between clinical action types and corresponding rule sets can be accessed to select the appropriate rule set for evaluation. For example, a request for an imaging procedure can cause the rules engine to select guidelines specifying eligibility, prior results, or diagnostic codes relevant to the imaging procedure.

At 715, the method can include generating a query for selecting data relevant to the type of clinical action and/or the request. For example, information regarding the type of clinical action can be used to structure the query for the chunk of atomic units. Attributes of the clinical action, such as diagnostic codes, procedure codes, or referenced guideline identifiers, can be identified, and can be mapped to relational fields associated with the atomic unit content and/or atomic unit attributes in the database. In some implementations, the one or more filters can be generated to correspond to metadata columns, such as patient identifiers, document types, or temporal ranges derived from the request. For example, a filter can be included to select atomic units that represent clinical notes tied to a specific patient identifier and date interval, or a filter referencing guideline document atoms that include relevant procedure terminology. A relational expression can be generated that specifies a chunk type, such as a paragraph, page, or section, according to atomic attributes linked to the type of clinical action identified from the request.

At 720, the method can include retrieving a group of atomic units from a relational database based on the query. For example, the query can be applied to the database to cause retrieval of the group (e.g., chunk) of atomic units.

At 725, the method can include determining that the rules are satisfied based on data (e.g., values, attributes) of the retrieved group of atomic units. For example, data values within the retrieved atomic units can be identified as corresponding to parameters specified in the applicable rules. In some implementations, one or more conditions can be specified by each rule based on attribute fields such as clinical code, date, or confidence score of the retrieved atomic units. For example, a determination can be made that the atomic units satisfy a rule based on the values of the atomic units being determined (e.g., using the rules) to meet threshold conditions or pattern matches defined by the rule expressions.

At 730, the method 700 can include authorizing the clinical action based on the rules being satisfied. For example, an output can be generated and/or transmitted that indicates the authorization. The output can be sent to a system that provided the request for the authorization of the clinical action. The output can be generated to include an audit trail indicating at least some of the data or evidence used to authorize the clinical action.

Systems and methods as described herein can be implemented by any of various neural networks and/or machine learning models. These can include, for example and without limitation, one or more neural networks (or layers, nodes, weights, and/or biases thereof), convolutional neural networks, recurrent neural networks, attention networks, transformer networks, encoders, decoders, sequence to sequence models, generative models, pretrained models, diffusion models, multimodal models, generative adversarial networks, or various combinations thereof, which may be configured (e.g., trained, fine-tuned, having transfer learning performed, updated or operated by in-context learning, examples, or prompting, etc.) through operations such as supervised learning, self-supervised learning, or unsupervised learning. Systems and methods as described herein can be implemented in any of various artificial intelligence architectures or processing pipelines, including, for example, agentic pipelines, retrieval-based pipelines (e.g., retrieval-augmented generation), or various combinations thereof.

Having now described some illustrative implementations, it is apparent that the foregoing is illustrative and not limiting, having been presented by way of example. In particular, although many of the examples presented herein involve specific combinations of method acts or system elements, those acts and those elements can be combined in other ways to accomplish the same objectives. Acts, elements and features discussed in connection with one implementation are not intended to be excluded from a similar role in other implementations or implementations.

The hardware and data processing components used to implement the various processes, operations, illustrative logics, logical blocks, modules and circuits described in connection with the implementations disclosed herein can be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor can be a microprocessor, or, any conventional processor, controller, microcontroller, soc (system on chip), som (system on module) or state machine. A processor also can be implemented as a combination of computing devices, such as a combination of a DSP and a microprocessor, a plurality of microprocessors, one or more microprocessors in conjunction with a DSP core, or any other such configuration. In some implementations, particular processes and methods can be performed by circuitry that is specific to a given function. The memory (e.g., memory, memory unit, storage device, etc.) can include one or more devices (e.g., RAM, ROM, Flash memory, hard disk storage, etc.) for storing data and/or computer code for completing or facilitating the various processes, layers and modules described in the present disclosure. The memory can be or include volatile memory or non-volatile memory, and can include database components, object code components, script components, or any other type of information structure for supporting the various activities and information structures described in the present disclosure. According to an exemplary implementation, the memory is communicably connected to the processor via a processing circuit and includes computer code for executing (e.g., by the processing circuit and/or the processor) the one or more processes described herein.

The present disclosure contemplates methods, systems and program products on any machine-readable media for accomplishing various operations. The implementations of the present disclosure can be implemented using existing computer processors, or by a special purpose computer processor for an appropriate system, incorporated for this or another purpose, or by a hardwired system. Implementations within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular can also embrace implementations including a plurality of these elements, and any references in plural to any implementation or element or act herein can also embrace implementations including only a single element. References in the singular or plural form are not intended to limit the presently disclosed systems or methods, their components, acts, or elements to single or plural configurations. References to any act or element being based on any information, act or element can include implementations where the act or element is based at least in part on any information, act, or element.

Any implementation disclosed herein can be combined with any other implementation or implementation, and references to “an implementation,” “some implementations,” “one implementation” or the like are not necessarily mutually exclusive and are intended to indicate that a particular feature, structure, or characteristic described in connection with the implementation can be included in at least one implementation or implementation. Such terms as used herein are not necessarily all referring to the same implementation. Any implementation can be combined with any other implementation, inclusively or exclusively, in any manner consistent with the aspects and implementations disclosed herein.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Systems and methods described herein can be embodied in other specific forms without departing from the characteristics thereof. Further relative parallel, perpendicular, vertical or other positioning or orientation descriptions include variations within +/−10% or +/−10 degrees of pure vertical, parallel or perpendicular positioning. References to “approximately,” “about” “substantially” or other terms of degree include variations of +/−10% from the given measurement, unit, or range unless explicitly indicated otherwise. Coupled elements can be electrically, mechanically, or physically coupled with one another directly or with intervening elements. Scope of the systems and methods described herein is thus indicated by the appended claims, rather than the foregoing description, and changes that come within the meaning and range of equivalency of the claims are embraced therein.

The term “coupled” and variations thereof includes the joining of two members directly or indirectly to one another. Such joining can be stationary (e.g., permanent or fixed) or moveable (e.g., removable or releasable). Such joining can be achieved with the two members coupled directly with or to each other, with the two members coupled with each other using a separate intervening member and any additional intermediate members coupled with one another, or with the two members coupled with each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling can be mechanical, electrical, or fluidic.

References to “or” can be construed as inclusive so that any terms described using “or” can indicate any of a single, more than one, and all of the described terms. A reference to “at least one of ‘A’ and ‘B’” can include only ‘A’, only ‘B’, as well as both ‘A’ and ‘B’. Such references used in conjunction with “comprising” or other open terminology can include additional items.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

References herein to the positions of elements (e.g., “top,” “bottom,” “above,” “below”) are merely used to describe the orientation of various elements in the FIGURES. The orientation of various elements can differ according to other exemplary implementations, and that such variations are intended to be encompassed by the present disclosure.

What is claimed is:

1. A system comprising:
one or more processors to:
establish a relational database that includes a plurality of atomic units extracted from a plurality of documents relating to one or more clinical actions, based at least on a type of modality of each document;
receive a request for authorization of a clinical action;
determine, using a rules engine, one or more rules for a type of the clinical action;
submit a query to the relational database to retrieve, from the relational database, a group of atomic units dynamically identified as corresponding to the type of clinical action and based on one or more filters relating to the type of the clinical action;
determine, by the rules engine using the one or more rules, that data of the group of atomic units resulting from the query satisfies the one or more rules; and
authorize the clinical action responsive to the determination.

2. The system of claim 1, wherein the one or more processors are to select at least one of the rules engine or the one or more rules based on at least one of the type of the clinical action or the group of atomic units.

3. The system of claim 1, wherein the one or more processors are to formulate the one or more filters for the query to include at least one filter related to the type of the clinical action.

4. The system of claim 1, wherein the one or more processors are to retrieve the group of atomic units to include atomized content from one or more documents of the plurality of documents from which the group of atomic units are extracted and metadata of the group of atomic units.

5. The system of claim 1, wherein the one or more processors are to:
generate, using the rules engine, a candidate determination that the group of atomic units resulting from the query satisfy the one or more rules;
present, using a user interface, an indication of the candidate determination;
receive, via the user interface, a confirmation of the candidate determination; and
authorization the clinical action based on the confirmation.

6. The system of claim 1, wherein the one or more processors are to:
extract, from a given document of the plurality of documents, each of a first atomic unit comprising a token representing text and a second atomic unit comprising a pixel of an image;
assign a first position attribute to the first atomic unit indicating a position of the text in the given document; and
assign a second position attribute to the second atomic unit indicating a position of the pixel in the document.

7. The system of claim 1, wherein the one or more processors are to define the one or more filters to select one or more medical records regarding a patient for which to authorize the clinical action.

8. The system of claim 1, wherein the clinical action comprises at least one of a test to perform for a patient, a treatment to provide to the patient, or an appointment to schedule between the patient and a provider.

9. The system of claim 1, wherein the plurality of documents comprise at least one of a medical record, diagnostic imaging data, a test result, or claims data.

10. The system of claim 1, wherein the one or more processors are to:
provide the data of the group of atomic units to at least one machine learning model to cause the machine learning model to update the data to have at least one of increased precision or increased recall; and
determine that the one or more rules are satisfied based on the updated data.

11. The system of claim 1, wherein the plurality of documents comprise a guideline document regarding the clinical action, and the one or more processors are to at least one of:
select the one or more rules according to an atomic unit corresponding to the guideline document; or
generate the query, according to the one or more rules, to select at least a portion of the guideline document.

12. The system of claim 1, wherein the one or more rules identify the one or more filters.

13. The system of claim 1, wherein the one or more processors are to generate audit data regarding the determination, the audit data comprising content of at least one atomic unit of the group of atomic units and a location of the content in a corresponding document of the plurality of documents from which the at least one atomic unit is extracted.

14. A method, comprising:
receiving, by one or more processors, a request for authorization of a clinical action;
determining, using a rules engine, one or more rules for a type of the clinical action;
inputting, by the one or more processors, the query to the relational database to retrieve data of a group of the plurality of atomic units, the group dynamically identified as corresponding to the type of the clinical action and based on one or more filters;
determining, by the rules engine using the one or more rules, that the one or more rules are satisfied based at least on the retrieved data; and
authorizing the clinical action responsive to the determination.

15. The method of claim 14, further comprising receiving the request from a clinical system remote from the one or more processors, and transmitting an indication of the authorization to the clinical system.

16. The method of claim 14, comprising:
generating, using the rules engine, a candidate determination that the group of atomic units resulting from the query satisfy the one or more rules;
receiving, via a user interface, a confirmation of the candidate determination; and
outputting the authorization of the clinical action based on the confirmation.

17. The method of claim 14, comprising generating the one or more filters, based on the one or more rules, to query for notes regarding a previous clinical interaction with a patient associated with the request and for a protocol for the clinical action.

18. The method of claim 14, comprising generating the one or more filters to select one or more medical records regarding a patient for which to authorize the clinical action.

19. The method of claim 14, wherein the plurality of documents comprise at least one of a medical record, diagnostic imaging data, a test result, or claims data.

20. A non-transitory computer-readable medium comprising machine-readable instructions that when executed by one or more processors, cause the one or more processors to execute operations comprising:
updating a relational database to include a plurality of atomic units extracted from a plurality of documents relating to a clinical action, based at least on a type of modality of each document;
receiving a request for authorization of the clinical action;
determining, using a rules engine, one or more rules for a type of the clinical action;
inputting a query to the relational database to retrieve, from the relational database, a group of atomic units dynamically identified as corresponding to the type of clinical action and based on one or more filters of the query;
determining, by the rules engine using the one or more rules, that data of the group of atomic units resulting from the query satisfies the one or more rules; and
transmitting an authorization of the clinical action responsive to the determination.
